(19) United States
(12) Patent Application Publication
(10) Pub. No.: US 2002/0120136 A1
(43) Pub. Date: Aug. 29, 2002

(54) Method of preparing cephalosporins using 4-hydroxyphenylglycine derivatives

(76) Inventors: Gwan-Sun Lee, Seoul (KR); Jae-Heon Lee, Yongin-si (KR); Young-Kil Chang, Seoul (KR); Hong-Sun Kim, Seoul (KR); Chul-Hyun Park, Seongnam-si (KR); Gha-Seung Park, Goyang-si (KR); Cheol-Kyung Kim, Namyangju-si (KR)

Correspondence Address: David A. Einhorn, Esq., Anderson Kill & Olick, P.C., 1251 Avenue of the Americas, New York, NY, 10020, US

(21) Appl. No.: 10/083,777

(22) Filed: Feb. 25, 2002

(30) Foreign Application Priority Data
Feb. 26, 2001 (KR) 2001-9731
Feb. 26, 2001 (KR) 2001-9732
Feb. 26, 2001 (KR) 2001-9740
Feb. 26, 2001 (KR) 2001-9743

(51) Int. Cl. C 07D 5 1/16

(52) U.S. Cl. 540/217

(57) ABSTRACT

High purity cephalosporin compound can be easily prepared in a high yield by a process comprising the steps of: reacting a cephem compound with a 4-hydroxyphenylglycine derivative.

### FIELD OF THE INVENTION

The present invention relates to a method of preparing cephalosporin antibiotics; and to novel intermediates used in said method.

### BACKGROUND OF THE INVENTION

Cephalosporin antibiotics such as cefatrizine, cefadroxil and cefprozil are generally prepared by reacting a cephem derivative with a reactive derivative of 4-hydroxyphenylglycine such as: a reactive ester, e.g., mercaptobenzothiazolyl ester, phosphonate ester and thiophosphonate ester; a reactive amide, e.g., benzotriazolyl amide; an active compound, e.g., imidazolide and triazolide; and a mixed-acidic anhydride. However, in case such reactive ester or amide is used, it is difficult to obtain the desired product in a high purity form due to the occurrence of side-reactions as well as racemization. Such conventional methods are also hampered by other difficulties.

For example, the method disclosed in U.S. Pat. Nos. 4,520,022 and 4,699,979 comprises the steps of: protecting the amino group of 4-hydroxyphenylglycine; reacting with a cephem compound in the presence of dicyclohexylcarbodiimide, a condensing agent; and removing the protecting group to obtain cefprozil. However, both methods have the problems that the use of moisture-sensitive dicyclohexylcarbodiimide requires a rigorous anhydrous condition and a low purity product is obtained due to the difficulty of removing residual dicyclohexylurea.

U.S. Pat. Nos. 4,649,079; 3,970,651; 3,985,747; and 4,160,863 and GB Patent No. 1,532,682 disclose methods that generally comprise the steps of: reacting 4-hydroxyphenylglycine with phosgene; adding gaseous hydrogen chloride to synthesize 4-hydroxyphenylglycine chloride hydrochloride; and reacting with a cephem compound to obtain cefatrizine, cefadroxil or cefprozil. However, these methods require hazardous phosgene and gaseous HCl, which are difficult to handle and cause environmental problem.

The method disclosed in International Publication No. WO 98/04732 comprises reacting 4-hydroxyphenylglycine with ethylene glycol to synthesize an ester which is reacted with a cephem compound in the presence of acylase to obtain cefprozil. However, this method requires the use of an excess amount of expensive enzyme and, therefore, is not suitable for mass-production.

The method disclosed in GB Patent No. 1,240,687 comprises reacting protected 4-hydroxyphenylglycine with ethyl chloroformate to obtain a carbonate derivative to be acylated with a cephem compound. However, this method gives a product of low purity.

Thus, there has been enormous efforts to develop an improved process for preparing a high purity cephalosporin.

### SUMMARY OF THE INVENTION

Accordingly, it is a primary object of the present invention to provide a high yield process for preparing a high purity cephalosporin.

It is another object of the present invention to provide novel intermediates that can be advantageously used in said method.

In accordance with one aspect of the present invention, there is provided a method of preparing a compound of formula (I) which comprises reacting a cephem compound of formula (II) with a 4-hydroxyphenylglycine derivative of formula (III) or formula (IV)

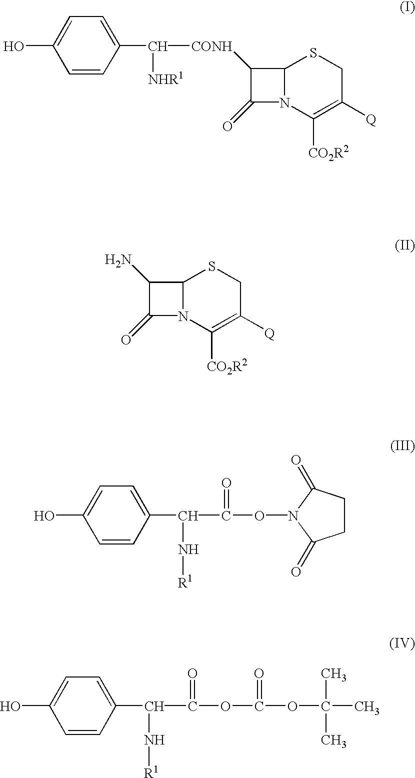

wherein R¹ is hydrogen or an amino protecting group,

R² is hydrogen or a carboxy protecting group, and

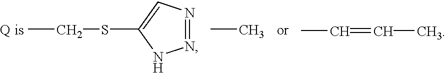

In accordance with another aspect of the present invention, there is provided 4-hydroxyphenylglycine derivatives of formula (III) and formula (IV) which are used in the above method:

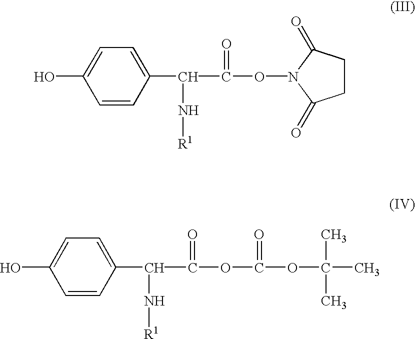

wherein R¹ is hydrogen or an amino protecting group.

### DETAILED DESCRIPTION OF THE INVENTION

The amino group in the cephalosporin compound of formula (I) may be protected with a common protecting group. The term “a common protecting group” as used herein refers to a protecting group which is conventionally used in cephalosporin-based compound; and exemplary protecting groups include fomyl, acetyl, chloroacetyl, benzyl, benzylidene, salicylidene, diphenylmethyl, triphenylmethyl, trichloroethoxycarbonyl, tetrahydropyranyl, t-butoxycarbonyl and carbobenzyloxy, wherein t-butoxycarbonyl, which can be easily removed by the action of an acid, is preferred. Further, exemplary carboxy protecting groups include alkyl esters such as methyl and t-butyl; alkoxyalkyl esters such as methoxymethyl; alkylthioalkyl esters such as methylthiomethyl; haloalkyl esters such as 2,2,2-trichloroethyl; and aralkyl esters such as benzyl, p-methoxybenzyl and diphenylmethyl, wherein p-methoxybenzyl which can be easily removed by the action of an acid is preferred.

The cephalosporin compound of formula (I) may be prepared by reacting a cephem compound of formula (II) with a 4-hydroxyphenylglycine derivative of formula (III) or formula (IV), the reaction involving the compound of formula (III) being carried out preferably in the presence of an acid.

The cephem compound of formula (II) used in the present invention may be prepared in accordance with any of the known methods (see U.S. Pat. Nos. 3,867,380, 3,489,752 and 4,520,022).

Exemplary solvents which may be suitably used in the present invention are methylene chloride, chloroform, carbon tetrachloride, acetonitrile, ethyl acetate, 1,4-dioxane, tetrahydrofuran or a mixture thereof, wherein methylene chloride and acetonitrile are preferred. The amount of the solvent used ranges from 5 to 30 volumes(v/w), preferably from 10 to 20 volumes(v/w) based on the amount of the cephem compound of formula (II).

Exemplary acids which may be suitably used in the inventive process involving the compound of formula (III) are formic acid, acetic acid, propionic acid, butyric acid, isobutyric acid, benzoic acid, methanesulfonic acid, benzensulfonic acid or p-toluenesulfonic acid, wherein isobutyric acid is preferred. The acid may be used in an amount ranging from 0.2 to 2.0 equivalents, preferably from 0.5 to 1.5 equivalents based on the amount of the cephem compound of formula (II).

The above reaction in accordance with the present invention may be performed at a temperature ranging from 0 to 50° C., preferably, from 10 to 30° C., for a period ranging from 3 to 20 hours.

In the inventive process, 4-hydroxyphenylglycine derivative of formula (III) or formula (IV) may be used in an amount ranging from 1.0 to 2.0 equivalents, preferably from 1.2 to 1.5 equivalents based on the amount of the cephem compound of formula (II).

The 4-hydroxyphenylglycine derivative of formula (III) used in the present invention may be prepared by reacting a compound of formula (V) with N,N-disuccinamidyl carbonate of formula (VI) in the presence of a base:

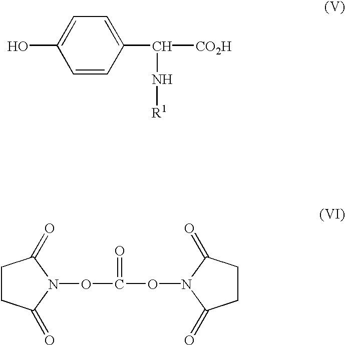

wherein R¹ is hydrogen or an amino protecting group.

The compound of formula (V) used in the above reaction may be prepared by protecting the amino group of 4-hydroxyphenylglycine with a protecting group which may be any of those conventionally used in cephalosporin synthesis, e.g., fomyl, acetyl, chloroacetyl, benzyl, benzylidene, salicylidene, diphenylmethyl, triphenylmethyl, trichloroethoxycarbonyl, tetrahydropyranyl, t-butoxycarbonyl and carbobenzyloxy, wherein t-butoxycarbonyl, which can be easily removed by the action of an acid, is preferred.

N,N-disuccinamidyl carbonate of formula (VI) may be prepared by reacting N-hydroxysuccinimide with triphosgene or trichloromethyl chloroformate (see Tetrahedron Letters, 49, 4745(1979)).

In the above reaction, N,N-disuccinamidyl carbonate may be used in an amount ranging from 1.0 to 3.0 equivalents, preferably from 1.2 to 2.2 equivalents, based on the amount of the compound of formula (V).

Exemplary solvents which may be suitably used in the above reaction are methylene chloride, chloroform, carbon tetrachloride, acetonitrile, ethyl acetate, 1,4-dioxane, tetrahydrofaran or a mixture thereof, wherein tetrahydrofuran is preferred. The amount of the solvent used may range from 5 to 30 volumes (v/w), preferably from 10 to 20 volumes (v/w), based on the amount of the compound of formula (V).

Exemplary bases which may be suitably used in the above reaction are triethylamine, n-tributylamine, N,N-dimethylaniline, pyridine, 1,4-diazabicyclo[2.2.2]octane, 1,5-diazabicyclo[4.3.0]non-5-ene, 1,8-diazabicyclo[5.4.0]undec-7-ene, N,N-dimethylaminopyridine or a mixture thereof, wherein N,N-dimethylaminopyridine is preferred. The base may be used in an amount ranging from 0.05 to 1.5 equivalents, preferably from 0.1 to 1.0 equivalent based on the amount of the compound of formula (V).

This reaction may be performed at a temperature ranging from 0 to 50° C., preferably from 10 to 30° C., for a period ranging from 3 to 5 hours.

Further, the 4-hydroxyphenylglycine derivative of formula (IV) used in the present invention may be prepared by reacting a compound of formula (V) with a pivaloyl halide of formula (VII) in the presence of a base:

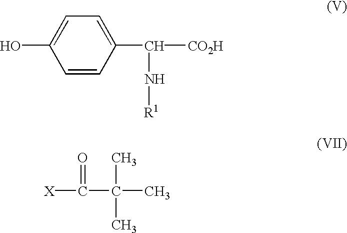

wherein,

R¹ is hydrogen or an amino protecting group, and

X is Cl, Br or 1.

In the above reaction, the pivaloyl halide of formula (VII) may be used in an amount ranging from 1.0 to 2.0 equivalents, preferably from 1.1 to 1.5 equivalents, based on the amount of the compound of formula (V). Exemplary solvents which may be suitably used in the above reaction are methylene chloride, chloroform, tetracarbonate chloride, acetonitrile, ethyl acetate, 1,4-dioxane, tetrahydrofuran or a mixture thereof. The amount of the solvent used may range from 3 to 15 volumes (v/w), preferably from 5 to 10 volumes(v/w), based on the amount of the compound of formula (V).

Exemplary bases which may be suitably used in the above reaction are triethylamine, n-tributylamine, N,N-dimethylaniline, pyridine, N,N-dimethylaminopyridine or a mixture thereof, wherein triethylamine is preferred. The base may be used in an amount ranging from 1.0 to 1.5 equivalents, preferably from 1.05 to 1.2 equivalents, based on the amount of the compound of the formula (V). This reaction may be performed at a temperature ranging from −10 to 10° C., preferably from 0 to 5° C., for a period ranging from 1 to 2 hours.

The inventive process described above is much simpler and entails a higher yield of a pure cephalosporin product as compared with any of the conventional methods.

The following Reference Examples and Examples are intended to further illustrate the present invention without limiting its scope; and the experimental methods used in the present invention can be practiced in accordance with the Reference Examples and Examples given below, unless otherwise stated.

Further, percentages given below for solid in solid mixture, liquid in liquid, and solid in liquid are on the bases of wt/wt, vol/vol and wt/vol, respectively, unless specifically indicated otherwise.

### Reference Example 1

Preparation of t-butoxycarbonylamino-(4-hydroxyphenyl) acetic acid (a compound of formula V)

33.4 g(0.20 mol) of 4-hydroxyphenylglycine and 87.3g(0.4 mol) of di-tert-butyl dicarbonate were dissolved in 340 ml of methanol, 41 ml(0.294 mol) of triethylamine was added thereto and then, stirred for 1 hour at 40° C. The resulting solution was cooled to room temperature and the solvent was removed under a reduced pressure. 400 ml of ethyl acetate and 100 ml of water were added to the residue, the resulting solution was adjusted to pH 3.0 with 5% HCl, and extracted with ethyl acetate. The organic layer was dried over anhydrous magnesium sulfate, filtered, and the solvent was removed under a reduced pressure. 200 ml of benzene was added to the residue and the mixture was stirred for 30 minutes. The solid formed was filtered and washed with benzene and dried in a vacuum, to obtain 45.8 g of the title compound as a white solid in a yield of 86%.

¹H-NMR (δ, DMSO-d₆): 1.37(9H,s,—OC(CH₃)₃), 4.94(1H,d,8.0 Hz,—CH), 6.69(2H,d,8.5Hz, benzene ring-H), 7.16(2H,d,8.5Hz, benzene ring-H), 9.42(1H, br s, NH), 12.57(1H, br s, COOH).

### Reference Example 2

Preparation of disuccinamidyl carbonate (a compound of formula (VI))

50 g(0.168 mol) of triphosgene and 97 g(0.842 mol) of N-hydroxysuccinimide were dissolved in 700 mn of tetrahydrofuran and cooled to 0° C. Added dropwise thereto was a mixture of 240 ml of tributylamine and 300 mf of tetrahydrofuran while maintaining the temperature at less than 5° C. Then, the resulting solution was stirred overnight at room temperature. The solid formed was filtered and washed with tetrahydrofuran, to obtain 96.1 g of the title compound as a white solid in a yield of 90%.

¹H-NMR (δ, DMSO-d₆): 2.88(4H,s,—CH₂CH₂—).

### Reference Example 3

Preparation of 2,5-dioxo-pyrrolidine-1-yl t-butoxycarbonyl-(4-hydroxyphenyl)acetate (a compound of formula(III))

10.7 g(40 mmol) of t-butoxycarbonylamino-(4-hydroxyphenyl)acetic acid obtained in Reference Example 1, 10.8 g(42 mol) of disuccinamidyl carbonate obtained in Reference Example 2 and 0.24 g(1.96 mmol) of N,N-dimethylaminopyridine were added to 100 ml of tetrahydrofuran and the mixture was stirred at room temperature for 5 hours. 20 ml of water was added thereto and the mixture was stirred overnight. Then, tetrahydrofuran was removed under a reduced pressure, 100 ml of ethyl acetate was added thereto and washed twice with 100 ml portions of saturated sodium bicarbonate solution, followed by washing twice with 100 ml portions of saturated NaCl solution. The organic layer was dried over anhydrous magnesium sulfate, filtered, the solvent was removed under a reduced pressure, and then, dried under a vacuum, to obtain 11.8 g of the title compound as a white solid in a yield of 80%.

¹H-NMR(δ, DMSO-d₆): 1.39(9H,s,—OC(CH₃)₃), 2.49(4H,s,—CH₂CH₂—), 5.46(1H,d,6.4 Hz, —CH), 6.73(2H,d,7.0 Hz, benzene ring-H), 7.27(2H,d,7 Hz, benzene ring-H), 7.98(1H, d,6.3Hz, NH), 9.58(1H,s,—OH)

IR(Cm⁻¹, KBr): 3335, 2980, 1736, 1518, 1369, 1208, 1163, 1090, 841, 650.561.

### Reference Example 4

Preparation of pivaloyl t-butoxycarbonylamino-(4-hydroxyphenyl)acetate (compound of formula(IV))

26.7 g(0.10 mol) of t-butoxycarbonylamino-(4-hydroxyphenyl)acetic acid obtained in Reference Example 1 was added to a mixture of 30 ml of N,N-dimethylformamide and 100 ml of methylene chloride and cooled to 0° C. Added thereto was 14.6 ml(0.105 mol) of triethylamine and the mixture was stirred for 10 minutes. Then, 12.9 ml(0.105 mol) of pivaloyl chloride in 70 ml of methylene chloride was added dropwise thereto over 20 minutes while maintaining the temperature at less than 5° C. The resulting solution was stirred at 0° C. for 30 minutes and the organic layer was washed three times with 100 ml portions of water. The organic layer was dried over anhydrous magnesium sulfate, filtered, the solvent was removed under a reduced pressure, and the residue was dried in a vacuum, to obtain 31.6 g of the title compound as a white solid in a yield of 95%.

¹H-NMR(δ, DMSO-d₆): 1.08(9H,s,pivaloyl C(CH₃)₃), 1.38(9H,s, —OC(CH₃)₃), 5.20(1H,d,7.5 Hz,—CH), 6.74(2H,d,8.5 Hz, benzene ring-H), 7.24(2H,d,8.5 Hz, benzene ring-H), 7.86(1H, d,7.4Hz,-NH), 9.56(1H,s, —OH)

IR(Cm⁻¹,KBr): 3451, 2980, 1804, 1701, 1513, 1173, 1055, 1021, 953.566

### EXAMPLE 1

Preparation of p-methoxybenzyl 7β-[D-2-(t-butoxycarbonylamino)-2(p-hydroxyphenyl)acetamido]-3-[propene- 1-yl]-3-cephem-4-carboxylate hydrochloride (a compound of formula (I))

2.0 g(5.04 mmol) of p-methoxybenzyl 7-amino-3-[propene-1 -yl]-3-cephem-4-carboxylate hydrochloride was dissolved in a mixture of 40 mL of ethylacetate and 40 ml of water and adjusted to pH 3.3 with saturated sodium bicarbonate solution. The organic layer was separated and dried over Ianhydrous magnesium sulfate, filtered, and the solvent was removed under a reduced pressure. 40 ml of acetonitrile was added to the oily residue and combined with 1.93 g(5.29 mmol) of 4-hydroxyphenylglycine derivative obtained in Reference Example 3 and 2 ml of isobutylic acid, and the mixture was stirred at room temperature overnight. Then, acetonitrile was removed under a reduced pressure and the resulting solution was dissolved in 40 ml portions of ethyl acetate and then, washed twice with 40 ml of saturated sodium bicarbonate solution, followed by washing twice with 40 ml of saturated NaCl solution. Then, the organic layer was dried over anhydrous magnesium sulfate, filtered, and the solvent was removed under a reduced pressure. The residue was dissolved in 6 ml of methanol, 1 mL of water was added dropwise thereto, stirred for 1 hour, and the solid formed was filtered, to obtain 2.88 g of the title compound as a white solid in a yield of 91%.

¹H-NMR(δ, DMSO-d₆): 1.37(9H,s,—OC(CH₃)₃), 1.47(3Hx 10/11,d,6.8 Hz,Z—CH₃), 1.74(3Hx1/11,d,5.7 Hz,E—CH₃), 3.74(3H,s, —OCH₃), 5.02˜5.19(4H,m,—CO₂CH₂CH—,6-H), 5.53˜5.60 (1H,m,vinyl H), 5.08˜5.72(1H,m,7-H), 6.01(1H,d,11.3 Hz,vinyl H), 6.65(2H,d,8.5 Hz, benzene ring-H), 6.91(2H,d,8.6 Hz, benzene ring-H), 7.19(2H,d,8.5 Hz, benzene ring-H), 7.34 (2H,d,8.6 Hz, benzene ring-H), 9.03(1H,d,8.4 Hz—NH), 9.34(1H,s,—OH).

### EXAMPLE 2

Preparation of 70 -[D-2-amino-2-(p-hydroxyphenyl)acetamido]-3-[propene-1-yl]-3-cephem-4-carboxylic acid (cefprozil)

10 g(16.4 mmol) of p-methoxybenzyl 7β-[D-2-(t-butoxycarbonylamino)-2-(p-hydroxyphenyl)acetamido]-3-[propene- 1 -yl]-3-cephem-4-carboxylate obtained in Example 1 was added to 100 ml of trifluoroacetic acid and the mixture was stirred at room temperature for 2 hours. Then, 200 ml of isopropylether was added dropwise thereto while maintaining the temperature at 5 to 10° C. The solid formed was filtered and washed with 100 ml of isopropylether and dried overnight under a vacuum. The resulting pale-yellow solid was suspended in 25 ml of methanol, 5.45 g(32.8 mmol) of sodium 2-ethylhexanoate in 350 ml of ethyl acetate was added thereto, and the mixture was stirred for 1 hour. The crystals formed were filtered, washed with 100 ml of ethyl acetate, dried under a vacuum. Sodium cefprozil thus obtained was dissolved in 30 ml of distilled water and adjusted to pH 3.5 to 3.7 with IN HC1. Then, the resulting solution was stirred for 30 minutes and further stirred for 30 minutes at 0° C. The solid formed was filtered, washed with 5 ml of chilled water, and dried under a vacuum, to obtain 5.41 g of the title compound as a pale yellow solid in a yield of 81%.

¹H-NMR(δ, D₂O+Na₂CO₃): 1.73(3HxIO/l l,d,6.5 Hz,Z—CH₃), 1.87(3Hx1/11,d,6.0 Hz,E—CH₃), 3.27˜3.60(2H,m,2-H), 5.13˜5.18(1H,d,4.5 Hz, 6-H), 5.22(1H,s,CHCO), 5.53˜6.03(1H,m, vinyl H),5.73(1H,d,4.5 Hz,7-H), 6.01(1H,d,11 Hz,vinyl H), 6.98(2H,d,9.0 Hz, benzene ring-H), 7.41(2H,d,8.8 Hz, benzene ring-H), 9.53 (1H,d,8.4 Hz, —NH).

### EXAMPLE 3

Preparation of p-methoxybenzyl 7P -[D-2-(t-butoxycarbonylamino)-2(p-hydroxyphenyl)acetamido]-3-[propene-1-yl]-3-cephem-4-carboxylate(a compound of formula (I))

2.0 g(5.04 mmol) of p-methoxybenzyl 7-amino-3-[propene-1-yl]-3-cephem-4-carboxylate hydrochloride was dissolved in a mixture of 40 ml of ethylacetate and 40 ml of water and adjusted to pH 3.3 with saturated sodium bicarbonate solution. The organic layer was separated and dried over anhydrous magnesium sulfate, filtered, and the solvent was removed under a reduced pressure. 40 ml of acetonitrile was added to the oily residue and 1.77 g(5.04 mmol) of 4-hydroxyphenyl anhydride obtained in Reference Example 4 was added thereto and stirred at room temperature for 4 hours. Then, after removing acetonitrile under a reduced pressure, 40 ml of ethyl acetate and 40 ml of water were added thereto. The organic layer was dried over anhydrous magnesium sulfate, filtered, and the solvent was removed under a reduced pressure. 10 ml of methanol was added thereto and the mixture was stirred for 30 minutes and the solid formed was filtered, to obtain 2.82 g of the title compound as a white solid in a yield of 89%.

¹H-NMR(δ, DMSO-d6): 1.37(9H,s,—OC(CH₃)₃), 1.47(3Hx10/1,d,6.8 Hz,Z—CH₃), 1.74(3Hx1/11,d,5.7 Hz,E—CH₃), 3.74(3H,s, —OCH₃), 5.02˜5.19(4H,m,—CO₂CH₂CH—,6-H), 5.53˜5.60 (1H,m,vinyl H), 5.08˜5.72(1H,m,7-H), 6.01(1H,d,11.3 Hz,vinylH), 6.65(2H,d,8.5 Hz, benzene ring-H), 6.91(2H,d,8.6 Hz, benzene ring-H), 7.19(2H,d,8.5 Hz, benzene ring-H), 7.34 (2H,d,8.6 Hz, benzene ring-H), 9.03(1H,d,8.4 Hz—NH), 9.34(1H,s,—OH).

### EXAMPLE 4

Preparation of 7β-[D-2-amino-2-(p-hydroxyphenyl)acetamido]-3-[propene-1-yl]-3-cephem-4-carboxylic acid (cefprozil)

10 g(16.4 mmol) of p-methoxybenzyl 7β-[D-2-(t-butoxycarbonylamino)-2-(p-hydroxyphenyl)acetamido]-3-[propene- 1-yl]-3-cephem-4-carboxylate obtained in Example 3 was added to 100 ml of trifluoroacetic acid and the mixture was stirred at room temperature for 2 hours. Then, 200 ml of isopropylether was added dropwise thereto while maintaining the temperature at 5 to 10° C. The solid formed was filtered, washed with 100 ml of isopropylether, and dried overnight under a vacuum. The pale-yellow solid thus obtained was suspended in 25 ml of methanol, 5.45 g(32.8 mmol) of sodium 2-ethylhexanoate dissolved in 350 ml of ethyl acetate was added thereto, and then, stirred for 1 hour. The crystals formed were filtered, washed with 100 ml of ethyl acetate, and dried under a vacuum. Sodium cefprozil thus obtained was dissolved in 30 ml of distilled water and adjusted to pH 3.5 to 3.7 with 1N HCl. The resulting solution was stirred for 30 minutes and then, further stirred for 30 minutes at 0° C. The solid formed was filtered, washed with 5 ml of chilled water and dried under a vacuum, to obtain 5.41 g of the title compound as a pale yellow solid in a yield of 81%.

H-NMR(δ, D₂O+Na₂CO₃): 1.73(3Hx10/11,d,6.5 Hz,Z—CH₃), 1.87(3Hx1/11,d,6.0 Hz,E—CH₃), 3.27˜3.60(2H,m,2-H), 5.13˜5.18(1H,d,4.5 Hz, 6-H), 5.22(1H,s,CHCO), 5.53˜6.03(1H,m, vinyl H),5.73(1H,d,4.5 Hz,7-H), 6.01(1H,d,11 Hz,vinyl H), 6.98(2H,d,9.0 Hz, benzene ring-H), 7.41(2H,d,8.8 Hz, benzene ring-H), 9.53 (1H,d,8.4 Hz, —NH).

While the invention has been described with respect to the above specific embodiments, it should be recognized that various modifications and changes may be made to the invention by those skilled in the art which also fall within the scope of the invention as defined by the appended claims.

What is claimed is:

1. A method of preparing a compound of formula (I) which comprises reacting a cephem compound of formula (II) with a 4-hydroxyphenylglycine derivative of formula (III) or formula (IV): 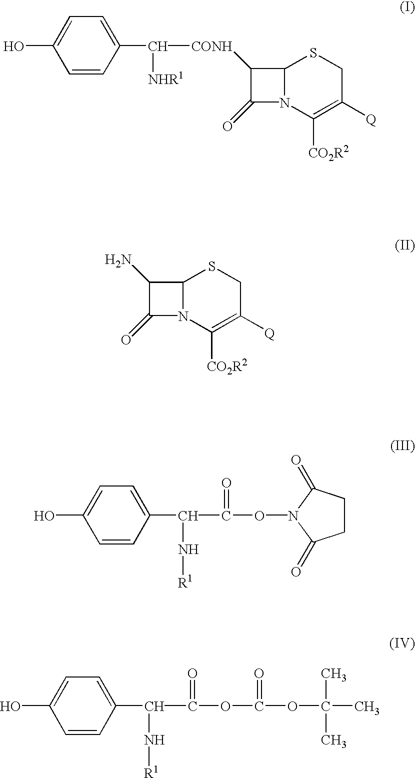
wherein R¹ is hydrogen or an amino protecting group,
R² is hydrogen or a carboxy protecting group, and 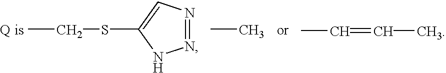
.

2. The method of claim 1, wherein the reaction involving the compound of formula (III) is conducted in the presence of an acid.

3. The method of claim 1, wherein the 4-hydroxyphenylglycine derivative is used in an amount ranging from 1.0 to 2.0 equivalents based on the amount of the cephem compound.

4. The method of claim 3, wherein the 4-hydroxyphenylglycine derivative is used in an amount ranging from 1.2 to 1.5 equivalents based on the amount of the cephem compound.

5. The method of claim 2, wherein the acid is formic acid, acetic acid, propionic acid, butyric acid, isobutyric acid, benzoic acid, methanesulfonic acid, benzenesulfonic acid, p-toluenesulfonic acid or a mixture thereof.

6. The method of claim 5, wherein the acid is isobutyric acid.

7. The method of claim 2, wherein the acid is used in an amount ranging from 0.2 to 2.0 equivalents based on the amount of the cephem compound.

8. The method of claim 7, wherein the acid is used in an amount ranging from 0.5 to 1.5 equivalents based on the amount of the cephem compound.

9. The method of claim 1, wherein the reaction is carried out at a temperature ranging from 0 to 50° C.

10. The method of claim 1, wherein the 4-hydroxyphenylglycine derivative of formula (III) is prepared by reacting a compound of formula (V) with N,N-disuccinamidyl carbonate of formula (VI) in the presence of a base: 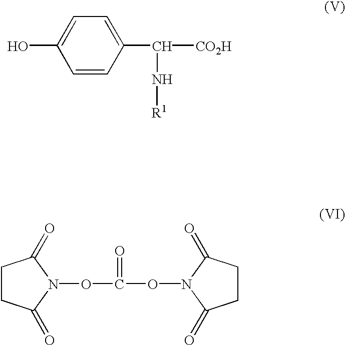
wherein R¹ is hydrogen or an amino protecting group.

11. The method of claim 10, wherein said N,N-disuccinamidyl carbonate is used in an amount ranging from 1.0 to 3.0 equivalents based on the amount of the compound of formula (V).

12. The method of claim 11, wherein said N,N-disuccinamidyl carbonate is used in an amount ranging from 1.2 to 2.2 equivalents based on the amount of the compound of formula (V).

13. The method of claim 10, wherein the base is triethylamine, n-tributylamine, N,N-dimethylaniline, pyridine, 1,4-diazabicyclo[2.2.2]octane, 1,5-diazabicyclo[4.3.0]non-5-ene, 1,8-diazabicyclo[5.4.0]undec-7-ene, N,N-dimethylaminopyridine or a mixture thereof.

14. The method of claim 10, wherein the base is used in an amount ranging from 0.05 to 1.5 equivalents based on the amount of the compound of formula (V).

15. The method of claim 14, wherein the base is used in an amount ranging from 0.1 to 1.0 equivalent based on the amount of the compound of formula (V).

16. The method of claim 10, wherein the reaction is carried out at a temperature ranging from 0 to 50° C.

17. The method of claim 1, wherein the 4-hydroxyphenylglycine derivative of formula (IV) is prepared by reacting the compound of formula (V) with a pivaloyl halide of formula (VII) in the presence of a base: 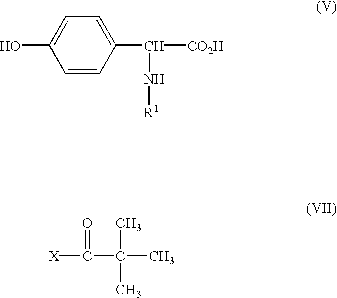
wherein, R¹ is hydrogen or an amino protecting group, and
X is Cl, Br or I.

18. The method of claim 17, wherein the pivaloyl halide is used in an amount ranging from 1.0 to 2.0 equivalents based on the amount of the compound of formula (V).

19. The method of claim 17, wherein the base is triethylamine, n-tributylamine, N,N-dimethylaniline, pyridine, N,N-dimethylaminopyridine or a mixture thereof.

20. The method of claim 17, wherein the base is used in an amount ranging from 1.0 to 1.5 equivalents based on the amount of the compound of formula (V).

21. The method of claim 17, wherein the reaction is carried out at a temperature ranging from −10 to 10° C.

22. 4-Hydroxyphenylglycine derivative of formula (III) which is used in the method of claim 1: 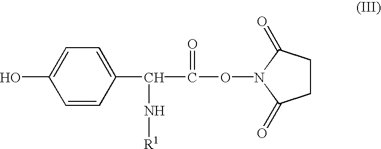
wherein R¹ is hydrogen or an amino protecting group.

23. 4-Hydroxyphenylglycine derivative of formula (IV) which is used in the method of claim 1: 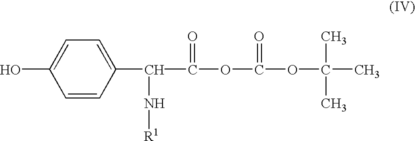
wherein R¹ is hydrogen or an amino protecting group.
